(19) United States
(12) Patent Application Publication
(10) Pub. No.: US 2011/0177570 A1
(43) Pub. Date: Jul. 21, 2011

(54) BACTERIAL MUTANTS FOR ENHANCED SUCCINATE PRODUCTION

(76) Inventors: Gino J.E. Baart, Hulst (NL); Joeri J.R. Beauprez, Bredene (BE); Maria R. Foulquié Moreno, Brussel (BE); Joseph . Heijnen, Rijen (NL); Jo Maertens, Gent (BE)

(21) Appl. No.: 12/737,303

(22) PCT Filed: Jun. 30, 2009
(86) PCT No.: PCT/EP2009/058173

§ 371 Date: Mar. 28, 2011

(30) Foreign Application Priority Data
Jul. 1, 2008 (EP) 08159442.6

(51) Int. Cl. C12P 7/46; C12N 1/00; C12N 1/21

(52) U.S. Cl. 435/145; 435/243; 435/252.33; 435/252.3

(57) ABSTRACT

The present invention relates to a method for obtaining enhanced metabolite production in micro-organisms, and to mutants and/or transformants obtained with said method. More particularly, it relates to bacterial mutants and/or transformants for enhanced succinate production, especially mutants and/or transformants that are affected in the import and export of succinate.

The present invention relates to a method for obtaining enhanced metabolite production in micro-organisms, and to mutants and/or transformants obtained with said method. More particularly, it relates to bacterial mutants and/or transformants for enhanced succinate production, especially mutants and/or transformants that are affected in the import and export of succinate.

Most environments are substrate limiting for micro-organisms, which has lead to very diverse and efficient carbon uptake systems (1). On the other hand, the excretion of end or intermediate products is less limiting for a micro-organism. Unless the excretion product has a competitive advantage (e.g. acetate excretion for acidification of the environment), excretion of certain end or intermediate products never needed to be as efficient, which has lead to a diverse selection of transport mechanisms (2,3).

From an industrial biotechnological perspective efficient excretion of an end-product can be a great advantage. It can lead to lower by-product formation, since the metabolism will not redirect carbon towards other exportable compounds and thus will lead to more easy to purify end-products. Additionally feedback inhibition of the pathway towards the product will be lowered, which logically leads to higher production rates. Both these production parameters, product purity and production rate, have previously been referred to as key parameters next to production yield (4-6) and were linked to the economically feasibility of a production process. The rising interest in industrial biotechnology originates in the increased awareness of the environmental impact of the existing industrial processes, the limited availability of fossil resources and the increasing political unrest that accompanies these evolutions. Up to now only few biotechnological processes are truly competitive with their chemical counterparts. In order to develop novel competitive processes a whole set of new techniques had to be developed, grouped in the so called discipline of ‘metabolic engineering’. This has already led to many new processes, in particular the development of succinate-production. Recent years many E. coli strains have been genetically modified with success, parallel to strain-development of Actinobacillus succinogenes, Mannheimia succiniciproducens, Anaerobiospirillum succiniciproducens.

Succinate as base chemical has first been pointed out by Greg Zeikus and coworkers in 1999 (7), after which the US Department of Energy (DOE) marked it as one of the top added value chemicals from renewable resources (4). Based on the petrochemical analogue, maleic anhydride, they have set the production price at [symbol] 0.45/kg. Nowadays, with the vastly increasing oil price, this analogue more than tripled in price. Herein lays the opportunity for bio-based chemicals to rise and become economical viable.

A second well defined parameter in the DOE report is the volumetric production rate, set at 2.5 g/l/h. These rates are not easily obtained. Low specific growth and production rates are thus far limiting to reach competitive succinic acid production, since high biomass concentrations are needed to obtain economical viable production rates.

A strategy that has never been tried before is pulling the metabolism towards a certain product instead of pushing it, leading to enhanced production rates. For this purpose the C4 transport systems lend themselves excellently.

A nice review on C4 dicarboxylic acid transport and sensors (8), groups the transporters in 5 large transporter families based on amino acid sequence similarities, the DctA family, the DcuAB family, DcuC family, CitT family and the TRAP family. This classification has been adopted and expanded by the transporter classification database, which summarizes all known transporters and membrane proteins (9) and has classified them in the class of the secondary transporters. All potential C4 dicarboxylic acid transporters are all then classified in 7 superfamilies: MFS, Dcu, DAACS, CSS, DASS, DcuC and AEC (3), of which the CSS superfamily does not have any representative in E. coli.

Looking more closely at the individual C4 dicarboxylic acid transport families, two main distinctions can be made, aerobic and anaerobic transport in Escherichia coli. While the DctA family mainly is operational in an aerobic environment, the DcuAB and DcuC family is operational in anaerobic conditions. Their function is closely related to the type of metabolism E. coli has in these conditions. Anaerobically, fumarate will function as a terminal electron acceptor, thus C4 dicarboxylic acids such as fumarate and malate will be interesting carbon sources for E. coli, while succinate is an end-product and will thus be preferably excreted (10). Transport in this condition will mainly be focussed on the import of fumarate, malate and other pathway intermediates and the export of succinate. Aerobically on the other hand, succinate is a crucial intermediate in the Krebs-cycle. It would thus be unfavourable for the cell to excrete succinate. In this case the cell is provided with a rather efficient succinate (C4-dicarboxylic acid) uptake system (DctA) which keeps the extracellular concentration low. It is also known that not only the DctA family, but a yet to be discovered carrier ensures the cell of succinate uptake (11). Enhancing succinate excretion would evidently mean, changing the whole expression scheme of these transporters.

Surprisingly, we found that by overexpression of the dcuC exporter gene, preferably overexpression under aerobic conditions, and by the knock out of the dctA importer gene, the production of succinate can be enhanced, especially of mutants that do have already a slightly higher succinate production.

A first aspect of the invention is a mutant and/or recombinant micro-organism comprising a genetic change leading to increased succinate export activity and decreased succinate import activity. A mutant as used here can be obtained by any method known to the person skilled in the art, including but not limited to UV mutagenesis and chemical mutagenesis. Some features may be obtained by classical mutagenesis, while others may be obtained by genetic engineering. Preferably the mutant strain is a recombinant strain, where all mutations are obtained by site directed mutagenesis and/or transformation. Preferably said mutant and/or recombinant is selected from a genus known to produce succinic acid. Even more preferably, said mutant and/or recombinant is an Escherichia coli strain.

Preferably, the genetic change in said mutant and/or recombinant strain is affecting in the dcuC exporter gene and the dctA importer gene, or in the orthologues thereof. Orthologues, as used here are genes in other genera, with a certain percentage identity at amino acid level, and a similar function. Preferably, said percentage identity, as measured by a protein BLAST, is at least 40%, even more preferably at least 50%, most preferably at least 60%. Beside the dcuC exporter gene and the dctA importer genes other importer of exporter genes might be affected.

Preferably, said genetic change is the replacement of the promoter of the dcuC exporter gene, and the knock out of the dctA importer gene. Even more preferably, the promoter of the dcuC exporter gene is replaced by a strong promoter, most preferably by a strong promoter functioning under aerobic conditions.

Preferably, the mutant and/or recombinant micro-organism, according to the invention, further comprises a genetic change in one or more of the genes selected from the group consisting of ackA, poxB, pta, arcA, sdhA, sdhB, sdhC, sdhD, iclR, citD, citE, citF, pckA, maeA, maeB, eda, edd gltA, ppc, sstT, ydjN, ygjE, citT/ybdS, ybhI, yfbS, yhjE and ydfJ. Possibly, other genes may be selected on the base of their importance in the metabolic network (Table II).

Another aspect of the invention is the use of a mutant and/or recombinant micro-organism comprising a genetic change leading to increased succinate export activity and/or decreased succinate import activity, in combination with a genetic change leading to increased succinate production to produce succinate. Increased succinate production is defined here as an increase in succinate productivity per unit of biomass or per unit of volume, and/or an increased extracellular succinate concentration, and/or an increase in succinate yield per unit of substrate. Preferably, said genetic change leading to increased succinate production is a genetic change in one or more of the genes selected from the group consisting of ackA, poxB, pta, arcA, sdhA, sdhB, sdhC, sdhD, iclR, citD, citE, citF, pckA, maeA, maeB, eda, edd gltA, ppc, sstT, ydjN, ygjE, citT/ybdS, ybhI, yfbS, yhjE and ydfJ. Possibly, other genes may be selected on the base of their importance in the metabolic network (Table II). Preferably, said use is the use under aerobic conditions.

Still another aspect of the invention is a mutant and/or recombinant micro-organism comprising a genetic change leading to increased succinate export activity and decreased succinate import activity for the production of succinate. Preferably, said mutant and/or recombinant micro-organism, further comprises a genetic change in one or more of the genes selected from the group consisting of ackA, poxB, pta, arcA, sdhA, sdhB, sdhC, sdhD, iclR, citD, citE, citF, pckA, maeA, maeB, eda, edd gltA, ppc, sstT, ydjN, ygjE, citT/ybdS, ybhI, yfbS, yhjE and ydfJ. Possibly, other genes may be selected on the base of their importance in the metabolic network (Table II).

### BRIEF DESCRIPTION OF THE FIGURES

FIG. 1: Gene knock out strategy (13) (top) and Gene knock in strategy (bottom)

FIG. 2: Construction of promoter delivery system for gene overexpression

FIG. 3: A: antibiotic resistance gene flanked with FRT sites, 50-nt homologies and restriction site regions; B and C: part of the gene of interest with the mutation; D: gene of interest with the mutation flanked by restriction site regions. 1: KO of the gene of interest; 2: mutant strain containing the point mutated gene of interest.

FIG. 4: Different succinate production rates (A) and yields (B) of E. coli MG1655 strains with modified C4-dicarboxylic acid transport: sdhAB: knock out of sdhAB; dcuC: overexpression of dcuC under control of promoter p37; dctA: knock out of dctA.

FIG. 5: Average growth rate of the wild type MG1655 and the dctA knock out strain under different conditions. The total amount of carbon is the same in each of the experiments (set to 0.5 c-mol/l). The p-values were obtained from a Student t test with 95% confidence interval.

FIG. 6: succinate yield in different genetic backgrounds. 0: wild type; * FNR: point mutation; 15: ΔpckA, 917: ΔmaeAB; 123467+: ΔackA Δpta ΔpoxB ΔiclR ΔarcA ΔsdhAB; 6: ΔarcA; 123467 20+: ΔackA Δpta ΔpoxB ΔiclR ΔarcA ΔsdhAB ΔdctA; 7: ΔsdhAB; 7B+: ΔsdhAB ΔFNR-pro37-dcuC; 7 20B+: ΔsdhAB ΔdctA ΔFNR-pro37-dcuC; 123467B+: ΔackA Δpta ΔpoxB ΔiclR ΔarcA ΔsdhAB ΔFNR-pro37-dcuC; 123467 20B+: ΔackA Δpta ΔpoxB ΔiclR ΔarcA ΔsdhAB ΔdctA ΔFNR-pro37-dcuC; 123467 20B+ edd: ΔackA Δpta ΔpoxB ΔiclR ΔarcA ΔsdhAB ΔdctA ΔFNR-pro37-dcuC Δedd. The error bars show the standard deviation of at least five measurements in two fermentations.

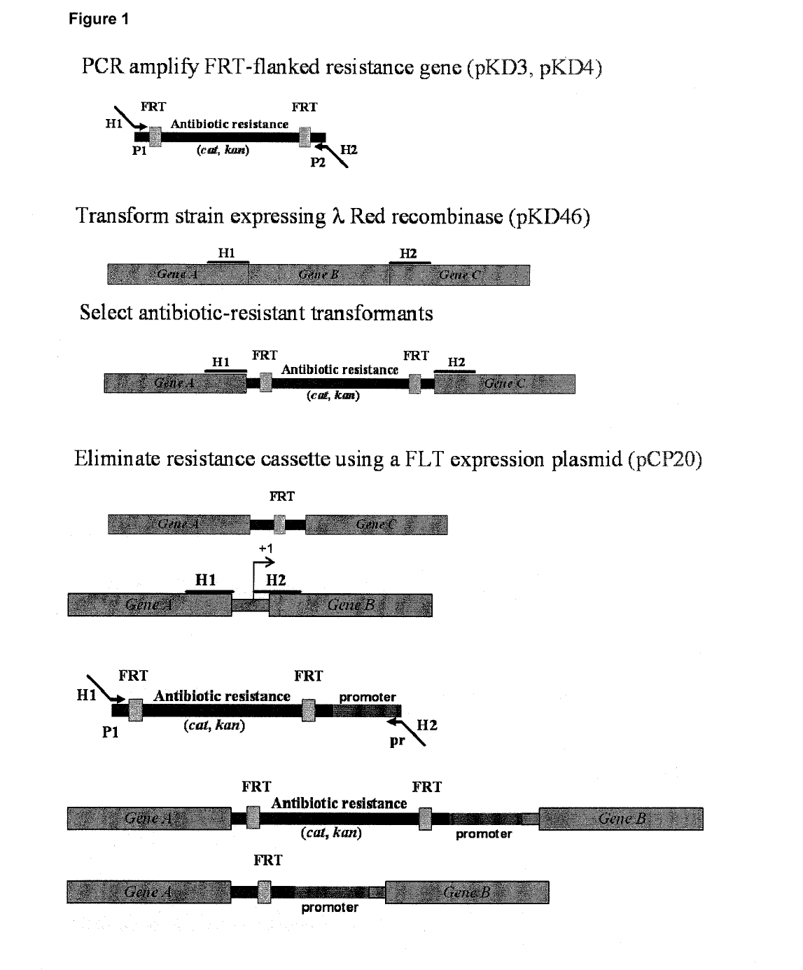

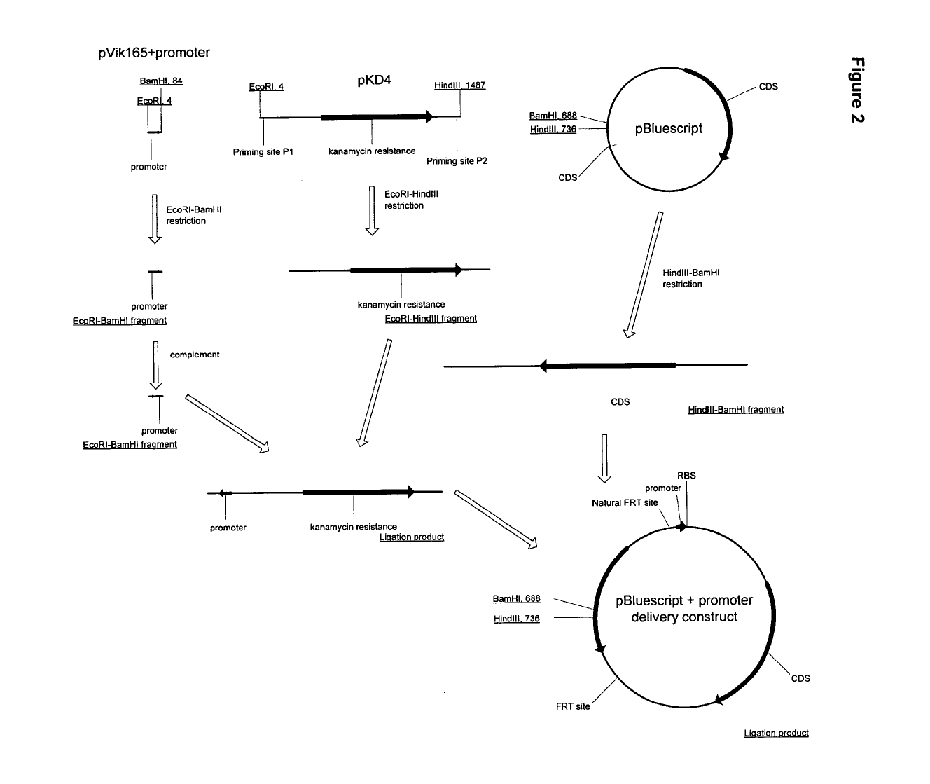

### EXAMPLES

Materials and Methods to the Examples

### Strains

Escherichia coli MG1655 [λ⁻, F⁻, rph-1] was obtained from the Coli Genetic Stock Center (CGSC). It was explicitly checked to not have the fnr deletion, as some strains with this name have it (12). The different strains were preserved in 50% glycerol-LB growth medium solution.

Table 1 summarizes all used strains, with their respectively mutations

TABLE I
Summary of all constructed strains
Strains based in MG1655

FNR*
ΔpckA
ΔmaeAB
ΔackA Δpta ΔpoxB ΔiclR ΔarcA ΔsdhAB
ΔarcA
ΔackA Δpta ΔpoxB ΔiclR ΔarcA ΔsdhAB ΔdctA
ΔsdhAB
ΔsdhAB ΔFNR-pro37-dcuC
ΔsdhAB ΔdctA
ΔsdhAB ΔdctA ΔFNR-pro37-dcuC
ΔackA Δpta ΔpoxB ΔiclR ΔarcA ΔsdhAB ΔFNR-pro37-dcuC
ΔackA Δpta ΔpoxB ΔiclR ΔarcA ΔsdhAB ΔdctA ΔFNR-pro37-dcuC
ΔackA Δpta ΔpoxB ΔiclR ΔarcA ΔsdhAB ΔdctA ΔFNR-pro37-dcuC Δedd
ΔackA Δpta ΔpoxB ΔiclR ΔarcA ΔsdhAB ΔdctA ΔFNR-pro37-dcuC Δedd ΔcitDEF
ΔackA Δpta ΔpoxB ΔiclR ΔarcA ΔsdhAB ΔdctA ΔFNR-pro37-dcuC Δedd ΔcitDEF ppc*
ΔackA Δpta ΔpoxB ΔiclR ΔarcA ΔsdhAB ΔdctA ΔFNR-pro37-dcuC Δedd Δeda ΔcitDEF ppc*
ΔackA Δpta ΔpoxB ΔiclR ΔarcA ΔsdhAB ΔdctA ΔFNR-pro37-dcuC Δedd Δeda ΔcitDEF ppc*
gltA*

### Media

The Luria Broth (LB) medium consisted of 1% tryptone peptone (Difco, Erembodegem, Belgium), 0.5% yeast extract (Difco) and 0.5% sodium chloride (VWR, Leuven, Belgium). Shake flask medium contained 2 g/l NH₄Cl, 5 g/l (NH₄)₂SO₄, 2.993 g/l KH₂PO₄, 7.315 g/l K₂HPO₄, 8.372 g/l MOPS, 0.5 g/l NaCl, 0.5 g/l MgSO₄.7H₂O, 16.5 g/l glucose.H₂O, 1 ml/l vitamin solution, 100 μl/l molybdate solution and 1 ml/l selenium solution. The medium was set to a pH of 7 with 1M of KH₂PO₄.

Vitamin solution consisted of 3.6 g/l FeCl₂.4H₂O, 5 g/l CaCl₂.2H₂O, 1.3 g/l MnCl₂.2H₂O, 0.38 g/l CuCl₂.2H₂O, 0.5 g/l CoCl₂.6H₂O, 0.94 g/l ZnCl₂, 0.0311 g/l H₃BO₄, 0.4 g/l Na₂EDTA.2H₂O and 1.01 g/l thiamine.HCl. The molybdate solution contained 0.967 g/l Na₂Moa₄.2H₂O. The selenium solution contained 42 g/l SeO₂. The minimal medium during fermentations contained 6.75 g/l NH₄Cl, 1.25 g/l (NH₄)₂SO₄, 1.15 g/l KH₂PO₄, 0.5 g/l NaCl, 0.5 g/l MgSO₄.7H₂O, 16.5 g/l glucose.H₂O, 1 ml/l vitamin solution, 100 μl/l molybdate solution and 1 ml/l selenium solution with the same composition as described above.

### Cultivation Conditions

A preculture from a single colony on a LB-plate was started in 5 ml LB medium during 8 hours at 37° C. on an orbital shaker at 200 rpm. From this culture, 2 ml was transferred to 100 ml minimal medium in a 500 ml shake flask, and incubated for 16 hours at 37° C. on an orbital shaker at 200 rpm. 4% inoculum was used in a 2 l Biostat B culture vessel with 1.5 l working volume (Sartorius-Stedim Biotech SA, Melsungen, Germany). The culture conditions were: 37° C., stirring at 800 rpm, gas flow rate of 1.5 l/min. The pH was maintained at 7 with 0.5M H₂SO4 and 4M KOH. The exhaust gas was cooled down to 4° C. by an exhaust cooler (Frigomix 1000, Sartorius-Stedim Biotech SA, Melsungen, Germany). 10% solution of silicone antifoaming agent (BDH 331512K, VWR Int Ltd., Poole, England) was added when foaming rised during the fermentation (approx 10 μl). The off-gas was measured with an EL3020 off-gas analyser (ABB Automation GmbH, 60488 Frankfurt am Main, Germany).

### Sampling Methodology

The bioreactor contains in its interior a harvest pipe (BD Spinal Needle, 1.2×152 mm (BDMedical Systems, Franklin Lakes, N.J.—USA) connected to a reactor port, linked outside to a Masterflex 14 tubing (Cole-Parmer, Antwerpen, Belgium) followed by a harvest port with a septum for sampling. The other side of this Masterflex 16 tubing is connected back to the reactor vessel. This system is referred to as the rapid sampling loop. During sampling, reactor broth is pumped around in the sampling loop. It has been estimated that, at a flow rate of 150 ml/min, the reactor broth needs 0.04 s to reach the harvest port and 3.2 s to re-enter the reactor. At a pO2 level of 50%, there is around 3 mg/l of oxygen in the liquid. The pO2 level should never go below 20%. Thus 1.8 mg/l of oxygen may be consumed during transit through the harvesting loop. Assuming an oxygen uptake rate of 0.4 g oxygen/g biomass/h (the maximal oxygen uptake rate found at μₘₐₓ), this gives for 5 g/l biomass, an oxygen uptake rate of 2 g/l/h or 0.56 mg/l/s, which multiplied by 3.2 s (residence time in the loop) gives 1.8 mg/l oxygen consumption.

In order to stop the metabolism of cells during the sampling, reactor broth was sucked through the harvest port in a syringe filled with 62 g stainless steel beads precooled at −20° C., to cool down 5 ml broth immediately to 4° C.). Sampling was immediately followed by cold centrifugation (15000 g, 5 min, 4° C.). In the batch experiments, a sample for OD600 and extracellular measurements was taken each hour using the rapid sampling loop and the cold stainless bead sampling method. When exponential growth was reached, the sampling frequency was increased to every 20 minutes.

### Analytical Methods

Cell density of the culture was frequently monitored by measuring optical density at 600 nm (Uvikom 922 spectrophotometer, BRS, Brussel, Belgium). Cell dry weight was obtained by centrifugation (15 min, 5000 g, GSA rotor, Sorvall RC-5B, Goffin Meyvis, Kapellen, Belgium) of 20 g reactor broth in pre-dried and weighted falcons. The pellets were subsequently washed once with 20 ml physiological solution (9 g/l NaCl) and dried at 70° C. to a constant weight. To be able to convert OD measurements to biomass concentrations, a correlation curve of the OD to the biomass concentration was made.

The concentrations of glucose and organic acids were determined on a Varian Prostar HPLC system (Varian, Sint-Katelijne-Waver, Belgium), using an Aminex HPX-87H column (Bio-Rad, Eke, Belgium) heated at 65° C., equipped with a 1 cm precolumn, using 5 mM H2SO4 (0.6 ml/min) as mobile phase. Detection was done by a dual-wave UV-VIS (210 nm and 265 nm) detector (Varian Prostar 325) and a differential refractive index detector (Merck LaChrom L-7490, Merck, Leuven, Belgium). Peak identification was done by dividing the absorptions of the peaks in both 265 and 210 nm, which results in a constant value, typical for a certain compound (formula of Beer-Lambert).

### Genetic Methods

Plasmids were maintained in the host E. coli. DH5α (F⁻, φ80dlacZΔM15, Δ(lacZYA-argF)U169, deoR, recA1, endA1, hsdR17(rk⁻, mk⁺), phoA, supE44, λ⁻, thi-1, gyrA96, relA1), pKD46 (Red helper plasmid, Ampicillin resistance), pKD3 (contain an FRT-flanked chloramphenicol resistance (cat) gene), pKD4 (contains an FRT-flanked kanamycin resistance (kan) gene), and pCP20 (expresses FLP recombinase activity) plasmids were obtained from Prof. Dr. J-P Hernalsteens (Vrije Universiteit Brussel, Belgium). The plasmid pBluescript (Fermentas, St. Leon-Rot, Germany) was used to construct the derivates of pKD3 and pKD4 with a promoter library, or with alleles carrying a point mutation.

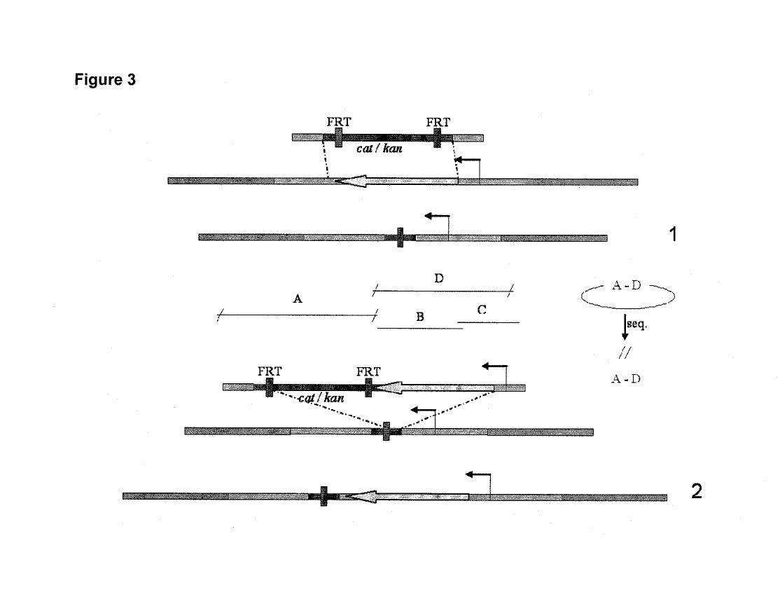

Mutations. The mutations consisted in gene disruption (knock-out, KO), replacement of an endogenous promoter by an artificial promoter (knock-in, KI), and point mutation (PM) (FIGS. 3). They were introduced using the concept of the Datsenko and Wanner (2000) (13) methodology.

Transformants carrying a Red helper plasmid were grown in 10-ml LB media with ampicillin (100 mg/L) and L-arabinose (10 mM) at 30° C. to an OD600 of 0.6. The cells were made electrocompetent by washing them with 50 ml of ice-cold water, a first time, and with 1 ml ice-cold water, a second time. Then, the cells were resuspended in 50 μl of ice-cold water.

Electroporation was done with 50 μl of cells and 10-100 ng of linear double-stranded-DNA product by using a Gene Pulser™ (BioRad) (600 OHMS, 25 μFD, and 250 volts). After electroporation, cells were added to 1-ml LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

Linear double-stranded-DNA. The linear ds-DNA amplicons were obtained by PCR using pKD3, pKD4 and their derivates as template. The primers used had a part of the sequence complementary to the template and another part complementary to the side on the chromosomal DNA where the recombination has to take place. For the KO, the region of homology was designed 50-nt upstream and 50-nt downstream of the start and stop codon of the gene of interest. For the KI, the transcriptional starting point (+1) had to be respected. The PM were generated with primers that contained the mutation. PCR products were PCR-purified, digested with DpnI, repurified from an agarose gel, and suspended in elution buffer (5 mM Tris, pH 8.0).

Elimination of the Antibiotic Resistance Gene. The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistances and of the FLP helper plasmid.

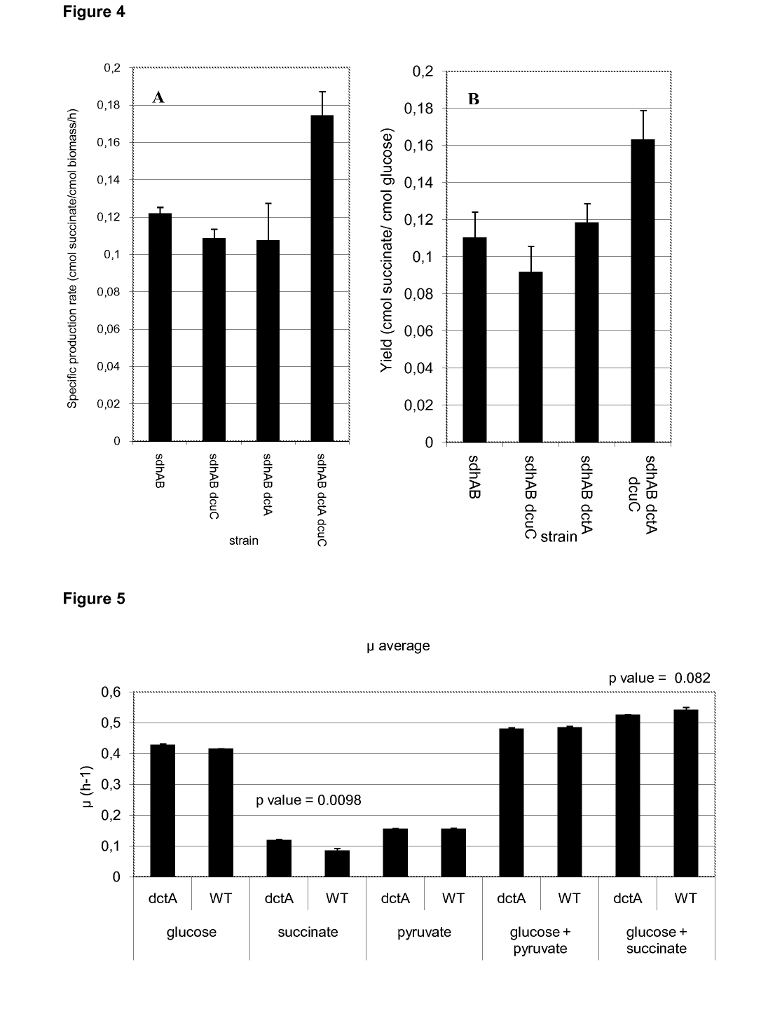

Point Mutations. The strategy consisted in two-steps, first a KO of the gene of interest and second to introduce the mutated gene in the same chromosomal location (FIG. 4). The gene of interest was amplified from the chromosomal DNA by PCR using primers containing the chosen mutation and flanked with restriction site regions. Two PCR products were generated from the same gene of interest, one from the promoter of the gene to 50-nt downstream of the mutation (C) and another from 50-nt upstream of the mutation to the stop codon (B). The mix of both PCR products was used as template to obtain the mutated gene flanked with restriction site regions (D). The antibiotic resistance genes (cat or kan) flanked with FRT sites were amplified from pKD3 or pKD4, respectively, by PCR with primers carrying the 50-nt homologies downstream of the stop codon of the gene of interest, the restriction site regions and 20-nt complementary to the template (A). The two PCR products A and D were digested with the appropriate restriction enzymes and introduced in a vector (p-Bluescript). After verifying the correct sequence of the gene, the inserted DNA was recovered by restriction enzyme digestion and used for further recombination.

### Mathematical Methods

### Metabolic Model

The metabolic network model of Lequeux et al. (2005) (14) was used. It includes glycolysis, with glucose transport by the phosphotransferase system (PTS), the pentose phosphate pathway, the Krebs cycle, and overflow metabolism. For each amino acid and nucleotide the anabolic reactions were included. Biosynthesis of lipopolysaccharides (LPS), lipid A, peptidoglycane, and the lipid bilayer are incorporated as well. The oxidative phosphorylation ratio (P/O) was set to 1.33 (15,16). The reactions and metabolites considered in the model are depicted in Tables 2 and 3 respectively.

### Partial Least Squares

Partial Least Squares (PLS) regression has been performed in the software package R (17). This generalization of multiple linear regression is able to analyze data with strongly collinear and numerous independent variables as is the case for the elementary flux modes under study. Partial least squares regression is a statistical method that links a matrix of independent variables X with a matrix of dependent variables Y, i.e., the flux ratios and the succinate yield, respectively. Therefore, the multivariate spaces of X and Y are transformed to new matrices of lower dimensionality that are correlated to each other. This reduction of dimensionality is accomplished by principal component analysis like decompositions that are slightly titled to achieve maximum correlation between the latent variables of X and Y (18).

### Elementary Flux Modes

The elementary flux modes of the stoichiometric E. coli model of Lequeux et al (2005) (14) were calculated by using Metatool 5 (19).

### Example 1

### Effect of Altered DctA and DcuC Activity in a sdhAB Knock Out Background

Three different promoters, P8, P37 and P55 were selected from a promoter bank. These P8, P37 and P55 are ranked from weak to strong. By evaluating in a chemostat, peculiarly enough higher acetate production rates were found in the strain with dcuC constitutively expressed with promoter P55 in comparison with the other promoters. Moreover, inclusion bodies were observed at the cellular poles of the dcuC-P55 strain. This leads to the conclusion that P55 is too strong as promoter, and the weaker P37 was used for further experiments.

The effect of the transporters was tested in an sdhAB knock out strain, which produces already some succinic acid. Neither enhanced production rate nor higher yield could be observed in strains in which solely DctA or DcuC activity was altered. The combination of altered import and export increased the specific production rate with about 55% and the yield with approximately 53% (FIG. 4).

Further investigation of the dctA single knock out has led to the conclusion that this strain grows faster on succinic acid than the wild type strain (FIG. 5). On glucose, pyruvate and the mixture of glucose and pyruvate the strains are growing equally fast. The experiment for the glucose-succinate mixture was repeated to determine a possible difference in growth rate of the two strains (in FIG. 4, there is a significant difference in case of 90% confidence, but not in case of 95% confidence). The results showed clearly that the two strains grow equally fast (p-value of 0.5). Only slight growth could be detected on fumarate, and no growth could be detected on malate.

### Example 2

### Effect of Altered DctA and DcuC Activity in Complex Genetic Backgrounds

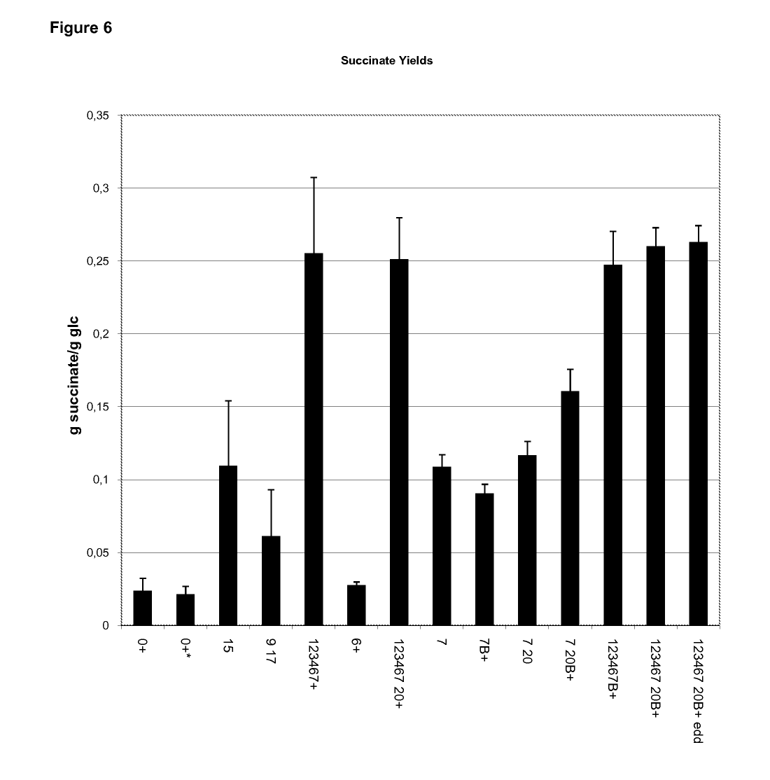

Different mutants affecting the succinate pathway have been constructed, as shown in Table I. These mutations were combined with the DctA knock out and the ΔFNR-pro37-dcuC overproducing construction. The results on the succinate yield are shown in FIG. 6.

TABLE II
Reactions of the metabolic network (14)

| | |
|---|---|
| HK: | ATP + GLC → ADP + G6P |
| PGI: | G6P [symbol] F6P |
| PFK: | ATP + F6P → ADP + FBP |
| ALD: | FBP [symbol] G3P + DHAP |
| TP1: | DHAP [symbol] G3P |
| G3PDH: | PiOH + NAD + G3P [symbol] NADH + H + BPG |
| PGK: | ADP + BPG [symbol] ATP + 3PG |
| PGM: | 3PG [symbol] 2PG |
| ENO: | 2PG [symbol] H2O + PEP |
| PyrK: | ADP + PEP → ATP + Pyr |
| PyrD: | NAD \| Pyr \| CoA → NADH \| H AcCoA \| CO2 |
| CitSY: | H2O + AcCoA + OAA → CoA + Cit |
| ACO: | Cit [symbol] iCit |
| CitDH: | NAD + iCit [symbol] NADH + H + CO2 + aKGA |
| AKGDH: | NAD + CoA + aKGA → NADH + H + CO2 + SucCoA |
| SucCoASY: | ADP + PiOH + SucCoA [symbol] ATP + CoA + Suc |
| SucDH: | FAD + Suc → FADH2 + Fum |
| FumHY: | H2O + Fum [symbol] Mal |
| MalDH: | NAD + Mal [symbol] NADH − H + OAA |
| iCitL: | iCit → Suc + Glyox |
| MalSY: | H2O \| AcCoA \| Glyox → CoA \| Mal |
| PEPCB: | H2O + PEP + CO2 → PiOH + OAA |
| PEPCBKN: | ATP + OAA → ADP + PEP + CO2 |
| PyrMalCB: | NAD + Mal → NADH + H + Pyr + CO2 |
| LacDH: | NADH + H + Pyr [symbol] NAD + Lac |
| PFLY: | Pyr + CoA → AcCoA + FA |
| EthDHLR: | 2NADH + 2H + AcCoA [symbol] 2NAD + CoA + Eth |
| AcKNLR: | ADP + PiOH + AcCoA [symbol] ATP + CoA + Ac |
| ActSY: | Pyr + Acdh → CO2 + Act |
| AcdhDH: | NADH + H + AcCoA [symbol] NAD + CoA + Acdh |
| EthDH: | NADH \| H \| Acdh [symbol] NAD \| Eth |
| Resp: | 1.33ADP + 1.33PiOH + NADH + H + 0.5O2 → 1.33ATP + |
| | NAD + 2.33H2O |
| H2CO3SY: | H2O + CO2 [symbol] H2CO3 |
| G6PDH: | NADP + G6P → NADPH + H + 6PGL |
| LAS: | H2O + 6PGL → 6PG |
| PGDH: | NADP + 6PG → NADPH + H + CO2 + Rl5P |
| PPI: | Rl5P [symbol] R5P |
| PPE: | Rl5P [symbol] Xu5P |
| TK1: | R5P + Xu5P [symbol] G3P + S7P |
| TA: | G3P + S7P [symbol] F6P + E4P |
| TK2: | Xu5P + E4P [symbol] F6P + G3P |
| FRPAS: | H2O + FBP → PiOH + F6P |
| R5P2R1P: | R5P [symbol] R1P |
| PTS: | GLC + PEP → G6P + Pyr |
| PPiOHHY: | PPiOH + H2O → 2PiOH |
| GluDH: | NADPH + H + aKGA + NH3 [symbol] NADP + H2O + Glu |
| GluLI: | ATP + NH3 + Glu → ADP + PiOH + Gln |
| GluSY: | NADPH + H + aKGA + Gln → NADP + 2Glu |
| AspSY: | ATP + H2O + Asp + Gln → AMP + PPiOH + Asn + Glu |
| AspTA: | OAA + Glu [symbol] aKGA + Asp |
| AspLI: | ATP + NH3 + Asp → AMP + PPiOH + Asn |
| AlaTA: | Pyr + Glu [symbol] aKGA + Ala |
| ValPyrAT: | Pyr + Val [symbol] aKIV + Ala |
| ValAT: | aKIV + Glu [symbol] aKGA + Val |
| LeuSYLR: | NAD + H2O + AcCoA + aKIV + Glu → NADH + H + CoA + |
| | CO2 + aKGA + Leu |
| aKIVSYLR: | NADPH + H + 2Pyr → NADP + H2O + CO2 + aKIV |
| IleSYLR: | NADPH + H + Pyr + Glu + Thr → NADP + H2O + CO2 + |
| | aKGA + NH3 + Ile |
| ProSYLR: | ATP + 2NADPH + 2H + Glu → ADP + PiOH + 2NADP + |
| | H2O + Pro |
| SerLR: | NAD + H2O + 3PG + Glu → PiOH + NADH + H + aKGA + |
| | Ser |
| SerTHM: | Ser + THF → H2O + Gly + MeTHF |
| H2SSYLR: | 2ATP + 3NADPH + ThioredH2 + 3H + H2SO4 → ADP + |
| | PPiOH + 3NADP + Thiored + 3H2O + H2S + PAP |
| PAPNAS: | H2O + PAP → AMP + PiOH |
| CysSYLR: | H2S + AcCoA + Ser → CoA + Cys + Ac |
| PrppSY: | ATP + R5P → AMP + PRPP |
| HisSYLR: | ATP + 2NAD + 3H2O + Gln + PRPP → 2PPiOH + PiOH + |
| | 2NADH + 2H + aKGA + His + AICAR |
| PheSYLR: | Glu + Chor → H2O + CO2 + aKGA + Phe |
| TyrSYLR: | NAD + Glu + Chor → NADH + H + CO2 + aKGA + Tyr |
| TrpSYLR: | Gln + Ser + Chor + PRPP → PPiOH + 2H2O + G3P + Pyr + |
| | CO2 + Glu + Trp |
| DhDoPHepAD: | H2O + PEP + E4P → PiOH + Dahp |
| DhqSY: | Dahp → PiOH + Dhq |
| DhsSYLR: | Dhq [symbol] H2O + Dhs |
| ShiSY: | NADPH + H + Dhs [symbol] NADP + Shi |
| ShiKN: | ATP + Shi → ADP + Shi3P |
| DhqDH: | NADPH + H + Dhq → NADP + Qa |
| ChorSYLR: | PEP + Shi3P → 2PiOH + Chor |
| DhsDH: | Dhs → H2O + ProtoCat |
| ProtoCatDC: | ProtoCat → CO2 + Cat |
| BkaSYLR: | H2O + O2 + Cat → Bka |
| GallicSY: | NAD + Dhs → NADH + H + Gallic |
| ThrSYLR: | ATP + H2O + HSer → ADP + PiOH − Thr |
| MDAPSYLR: | NADPH + H + Pyr + SucCoA + Glu + AspSA → NADP + |
| | CoA + aKGA + Suc + MDAP |
| LysSY: | MDAP → CO2 + Lys |
| MetSYLR: | H2O + SucCoA + Cys + MTHF − HSer → Pyr + CoA + Suc + |
| | NH3 + Met + THF |
| AspSASY: | ATP + NADPH + H + Asp → ADP + PiOH + NADP + |
| | AspSA |
| HSerDH: | NADPH + H + AspSA [symbol] NADP + HSer |
| CarPSY: | 2ATP + H2O + H2CO3 + Gln → 2ADP + PiOH + Glu + |
| | CarP |
| OrnSYLR: | ATP + NADPH + H + H2O + AcCoA + 2Glu → ADP + |
| | PiOH + NADP + CoA + aKGA + Orn + Ac |
| ArgSYLR: | ATP + Asp + Orn + CarP → AMP + PPiOH + PiOH + Fum + |
| | Arg |
| ThioredRD: | NADPH + Thiored + H [symbol] NADP + ThioredH2 |
| H2O2ox: | 2H2O2 → 2H2O + O2 |
| FAD2NAD: | NAD + FADH2 [symbol] NADH − FAD + H |
| CoQ2NAD: | NADH + CoQ + H [symbol] NAD + CoQH2 |
| NADH2NADPH | NADH + NADP [symbol] NAD + NADPH |
| AICARSYLR: | 6ATP + 3H2O + CO2 + Asp + 2Gln + Gly + FA + PRPP |
| | → 6ADP + PPiOH + 6PiOH + Fum + 2Glu + AICAR |
| IMPSYLR: | FTHF + AICAR → H2O + THF + IMP |
| AMPSYLR: | Asp + GTP + IMP → AMP + PiOH + Fum + GDP |
| AdKN: | ATP + AMP [symbol] 2ADP |
| ADPRD: | ADP + ThioredH2 → Thiored + H2O − dADP |
| dADPKN: | ATP + dADP → ADP + dATP |
| dADPPT: | H2O + dADP → PiOH + dAMP |
| IMPDH: | NAD + H2O + IMP → NADH + H + XMP |
| GMPSY: | ATP + H2O + Gln + XMP → AMP + PPiOH + Glu + GMP |
| GuKN: | ATP + GMP → ADP + GDP |
| GDPKN: | ATP + GDP → ADP + GTP |
| GDPRD: | ThioredH2 + GDP → Thiored + H2O + dGDP |
| dGDPKN: | ATP + dGDP → ADP + dGTP |
| dGDPPT: | H2O + dGDP → PiOH + dGMP |
| UMPSYLR: | O2 + Asp + PRPP + CarP → PPiOH + PiOH + H2O + CO2 + |
| | UMP + H2O2 |
| UrKN: | ATP + UMP → ADP + UDP |
| UDPKN: | ATP + UDP → ADP + UTP |
| CTPSY: | ATP + H2O + Gln + UTP → ADP + PiOH + Glu + CTP |
| CDPKN: | ATP + CDP [symbol] ADP + CTP |
| CDPPT: | H2O + CDP → PiOH + CMP |
| CMPKN: | ATP + CMP → ADP + CDP |
| CDPRD: | ThioredH2 + CDP → Thiored + H2O + dCDP |
| dCDPKN: | ATP + dCDP → ADP + dCTP |
| dCDPPT: | H2O + dCDP → PiOH + dCMP |
| dCTPDA: | H2O + dCTP → NH3 + dUTP |
| UDPRD: | ThioredH2 + UDP → Thiored + H2O + dUDP |
| dUDPKN: | ATP + dUDP → ADP + dUTP |
| dUTPPPAS: | H2O + dUTP → PPiOH + dUMP |
| dTMPSY: | MeTHF + dUMP → DHF + dTMP |
| dTMPKN: | ATP + dTMP → ADP + dTDP |
| dTDPKN: | ATP + dTDP → ADP + dTTP |
| dTDPPT: | H2O + dTDP → PiOH + dTMP |
| DHFRD: | NADPH + H + DHF → NADP + THF |
| FTHFSYLR: | NADP + H2O + MeTHF → NADPH + H + FTHF |
| GlyCA: | NAD + Gly + THF [symbol] NADH + H + CO2 + NH3 + MeTHF |
| MeTHFRD: | NADH + H + MeTHF → NAD + MTHF |
| FTHFDF: | H2O + FTHF → THF + FA |
| AcCoACB: | ATP + H2O + AcCoA + CO2 [symbol] ADP + PiOH + MalCoA |
| MalCoATA: | MalCoA + ACP [symbol] CoA + MalACP |
| AcACPSY: | MalACP → CO2 + AcACP |
| AcCoATA: | CoA + AcACP [symbol] AcCoA + ACP |
| C120SY: | 10NADPH + 10H + AcACP + 5MalACP → 10NADP + |
| | 5H2O + 5CO2 + C120ACP + 5ACP |
| C140SY: | 12NADPH + 12H + AcACP + 6MalACP → 12NADP + |
| | 6H2O + 6CO2 + C140ACP + 6ACP |
| C141SY: | 11NADPH + 11H + AcACP + 6MalACP → 11NADP + |
| | 6H2O + 6CO2 + C141ACP + 6ACP |
| C160SY: | 14NADPH + 14H + AcACP + 7MalACP → 14NADP + |
| | 7H2O + 7CO2 + C160ACP + 7ACP |
| C161SY: | 13NADPH + 13H + AcACP + 7MalACP → 13NADP + |
| | 7H2O + 7CO2 + C161ACP + 7ACP |
| C181SY: | 15NADPH + 15H + AcACP + 8MalACP → 15NADP + |
| | 8H2O + 8CO2 + C181ACP + 8ACP |
| AcylTF: | C160ACP + C181ACP + Go3P → 2ACP + PA |
| Go3PDH: | NADPH + H + DHAP [symbol] NADP + Go3P |
| DGoKN: | ATP + DGo → ADP + PA |
| CDPDGoSY: | CTP + PA [symbol] PPiOH + CDPDGo |
| PSerSY: | Ser + CDPDGo → CMP + PSer |
| PSerDC: | PSer → CO2 + PEthAn |
| GlnF6PTA: | F6P + Gln → Glu + GA6P |
| GlcAnMU: | GA6P [symbol] GA1P |
| NAGUrTF: | AcCoA + UTP + GA1P → PPiOH + CoA + UDPNAG |
| LipaSYLR: | ATP + 2CMPKDO + 2UDPNAG + C120ACP + 5C140ACP → |
| | ADP + 2CMP + UMP + UDP + 6ACP + Lipa + 2Ac |

TABLE III
Metabolites of the metabolic network (14)

| | | |
|---|---|---|
| 2PG | C₃H₇O₇P | 2-phophoglycerate |
| 3PG | C₃H₇O₇P | 3-phophoglycerate |
| 6PG | C₆H₁₃O₁₀P | 6-phosphogluconate |
| 6PGL | C₆H₁₁O₉P | 6-phosphogluconolacton |
| Ac | C₂H₄O₂ | Acetate |
| AcACP | C₂H₃OPept | Acetyl ACP |
| AcCoA | C₂₃H₃₄O₁₇N₇P₃S | Acetyl CoA |
| Acdh | C₂H₄O | Acetaldehyde |
| ACP | HPept | Acyl carier protein |
| Act | C₄H₈O₂ | Acetoine |
| ADP | C₁₀H₁₅O₁₀N₅P₂ | Adenosine diphosphate |
| ADPHEP | C₁₇H₂₇O₁₆N₅P₂ | ADP-Mannoheptose |
| AICAR | C₉H₁₅O₈N₄P | Amino imidazole carboxamide ribonucleotide |
| aKGA | C₅H₆O₅ | Alpha keto glutaric acid |
| aKIV | C₅H₈O₃ | Alpha-keto-isovalerate |
| Ala | C₃H₇O₂N | Alanine |
| AMP | C₁₀H₁₄O₇N₅P | Adenosine monophosphate |
| Ar5P | C₅H₁₁O₈P | Arabinose-5-phosphate |
| Arg | C₆H₁₄O₂N₄ | Arginine |
| Asn | C₄H₈O₃N₂ | Aspartate |
| Asp | C₄H₇O₄N | Asparagine |
| AspSA | C₄H₇O₃N | Aspartate semialdehyde |
| ATP | C₁₀H₁₆O₁₃N₅P₃ | Adenosine triphosphate |
| BGalAse | C_{4.98}H_{7.58}O_{1.5}N_{1.41} | Beta-galactosidase |
| | S_{0.0507} | |
| Biom | CH_{1.63}O_{0.392}N_{0.244} | Biomass |
| | P_{0.021}S_{0.00565} | |
| Bka | C₆H₈O₅ | Beta ketoadipate |
| BPG | C₃H₈O₁₀P₂ | 1-3-biphosphoglycerate |
| C120ACP | C₁₂H₂₃OPept | |
| C140ACP | C₁₄H₂₇OPept | |
| C141ACP | C₁₄H₂₅OPept | |
| C160ACP | C₁₆H₃₁OPept | |
| C161ACP | C₁₆H₂₉OPept | |
| C181ACP | C₁₈H₃₃OPept | |
| CarP | CH₄O₅NP | Carbamoyl phosphate |
| Cat | C₆H₆O₂ | Catechol |
| CDP | C₉H₁₅O₁₁N₃P₂ | Citidine diphosphate |
| CDPDGo | C₄₆H₈₃O₁₅N₃P₂ | CDP-diacylglycerol |
| CDPEthAn | C₁₁H₂₀O₁₁N₄P₂ | CDP-ethanolamine |
| Chor | C₁₀H₁₀O₆ | Chorismate |
| Cit | C₆H₈O₇ | cisaconitate |
| CL | C₇₇H₁₄₄O₁₆P₂ | Cardiolipin |
| CMP | C₉H₁₄O₈N₃P | Citidine monophosphate |
| CMPKDO | C₁₇H₂₆O₁₅N₃P | CMP-2-keto-3-deoxyoctanoate |
| CO2 | CO₂ | Carbondioxide |
| CoA | C₂₁H₃₂O₁₆N₇P₃S | Coenzyme A |
| CoQ | C₁₄H₁₈O₄ | Coenzyme Q, Ubiquinone (C5H8)n omitted |
| CoQH2 | C₁₄H₂₀O₄ | Ubiquinol |
| CTP | C₉H₁₆O₁₄N₃P₃ | Citidine triphosphate |
| Cys | C₃H₇O₂NS | Cysteine |
| dADP | C₁₀H₁₅O₉N₅P₂ | deoxy ADP |
| Dahp | C₇H₁₃O₁₀P | Deoxy arabino heptulosonate |
| dAMP | C₁₀H₁₄O₆N₅P | deoxy AMP |
| dATP | C₁₀H₁₆O₁₂N₅P₃ | deoxy ATP |
| dCDP | C₉H₁₅O₁₀N₃P₂ | deoxy CDP |
| dCMP | C₉H₁₄O₇N₃P | deoxy CMP |
| dCTP | C₉H₁₆O₁₃N₃P₃ | deoxy CTP |
| dGDP | C₁₀H₁₅O₁₀N₅P₂ | deoxy GDP |
| dGMP | C₁₀H₁₄O₇N₅P | deoxy GMP |
| DGo | C₃₇H₇₀O₅ | Diacyl glycerol |
| dGTP | C₁₀H₁₆O₁₃N₅P₃ | deoxy GTP |
| DHAP | C₃H₇O₆P | Dihydroxyaceton phosphate |
| DHF | C₁₉H₂₁O₆N₇ | Dihydrofolate |
| Dhq | C₇H₁₀O₆ | Dehydroquinate |
| Dhs | C₇H₈O₅ | Dehydroshikimate |
| DNA | C_{9.75}H_{14.2}O₇N_{3.75}P | DNA composition |
| dTDP | C₁₀H₁₆O₁₁N₂P₂ | deoxy TDP |
| dTMP | C₁₀H₁₅O₈N₂P | deoxy TMP |
| dTTP | C₁₀H₁₇O₁₄N₂P₃ | deoxy TTP |
| dUDP | C₉H₁₄O₁₁N₂P₂ | deoxy UDP |
| dUMP | C₉H₁₃O₈N₂P | deoxy UMP |
| dUTP | C₉H₁₅O₁₄N₂P₃ | deoxy UTP |
| E4P | C₄H₉O₇P | Erythrose-4-phosphate |
| Eth | C₂H₆O | Ethanol |
| F6P | C₆H₁₃O₉P | Ftuctose-6-phosphate |
| FA | CH₂O₂ | Formic Acid |
| FAD | C₂₇H₃₃O₁₅N₉P₂ | Flavine adeninen dinucleotide |
| FADH2 | C₂₇H₃₅O₁₅N₉P₂ | |
| FBP | C₆H₁₄O₁₂P₂ | Fructose-1-6-biphosphate |
| FTHF | C₂₀H₂₃O₇N₇ | Formyl tetrahydrofolate |
| Fum | C₄H₄O₄ | Fumarate |
| G1P | C₆H₁₃O₉P | Glucose-1-phosphate |
| G3P | C₃H₇O₆P | Glyceraldehyde-3-phosphate |
| G6P | C₆H₁₃O₉P | Glucose-6-phosphate |
| GA1P | C₆H₁₄O₈NP | D-glucosamine-6-phosphate |
| GA6P | C₆H₁₄O₈NP | D-glucosamine-6-phosphate |
| Gallic | C₇H₆O₅ | Gallic acid |
| GDP | C₁₀H₁₅O₁₁N₅P₂ | Guanosine diphosphate |
| GLC | C₆H₁₂O₆ | Glucose |
| Glcg | C₆H₁₀O₅ | Glycogen |
| Gln | C₅H₁₀O₃N₂ | Glutamine |
| Glu | C₅H₉O₄N | Glutamate |
| Gly | C₂H₅O₂N | Glycine |
| Glyox | C₂H₂O₃ | Glyoxylate |
| GMP | C₁₀H₁₄O₈N₅P | Guanosine monophosphate |
| Go3P | C₃H₉O₆P | Glycerol-3-phosphate |
| GTP | C₁₀H₁₆O₁₄N₅P₃ | Guanosine triphosphate |
| H | H⁺ | Hydrogene |
| H2CO3 | CH₂O₃ | Bicarbonate |
| H2O | H₂O | Water |
| H2O2 | H₂O₂ | |
| H2S | H₂S | Hydrogene sulfide |
| H2SO4 | H₂O₄S | Sulfuric acid |
| His | C₆H₉O₂N₃ | Histidine |
| HSer | C₄H₉O₃N | Homoserine |
| iCit | C₆H₈O₇ | isocitraat |
| Ile | C₆H₁₃O₂N | Isoleucine |
| IMP | C₁₀H₁₃O₈N₄P | Inosine monophosphate |
| Lac | C₃H₆O₃ | Lactate |
| Leu | C₆H₁₃O₂N | Leucine |
| Lipa | C₁₁₀H₁₉₆O₃₂N₂P₂ | Lipid A |
| Lipid | C_{40.2}H_{77.6}O_{8.41}N_{0.771} | Lipid composition |
| | P_{1.03} | |
| Lps | C₁₇₁H₂₉₈O₈₁N₄P₂ | Lipo Poly sacharide |
| Lys | C₆H₁₄O₂N₂ | Lysine |
| Mal | C₄H₆O₅ | Malate |
| MalACP | C₃H₃O₃Pept | Malonyl ACP |
| MalCoA | C₂₄H₃₄O₁₉N₇P₃S | Malonyl CoA |
| MDAP | C₇H₁₄O₄N₂ | Meso-diaminopimelate |
| Met | C₅H₁₁O₂NS | Methionine |
| MeTHF | C₂₀H₂₃O₆N₇ | Methyleen tetrahydro folate |
| MTHF | C₂₀H₂₅O₆N₇ | Methyl tetrahydrofolate |
| NAD | C₂₁H₂₈O₁₄N₇P₂⁺ | Nicotinamide adenine dinucleotide |
| NADH | C₂₁H₂₉O₁₄N₇P₂ | |
| NADP | C₂₁H₂₈O₁₇N₇P₃⁺ | Nicotinamide adenine dinucleotide phosphate |
| NADPH | C₂₁H₂₉O₁₇N₇P₃ | |
| NH3 | H₃N | Ammonia |
| O2 | O₂ | Oxygen |
| OAA | C₄H₄O₅ | Oxaloacetate |
| Orn | C₅H₁₂O₂N_{2.} | Ornithine |
| PA | C₃₇H₇₁O₈P | Phosphatidyl acid |
| PAP | C₁₀H₁₅O₁₀N₅P₂ | Phospho adenosine phosphate |
| PEP | C₃H₅O₆P | Phosphoenolpyruvate |
| Peptido | C₃₅H₅₃O₁₆N₇ | Peptidoglycane |
| PEthAn | C₃₉H₇₆O₈NP | Phosphatidyl ethanolamine |
| PG | C₄₀H₇₅O₉P | Phosphatidyl glycerol |
| Phe | C₉H₁₁O₂N | Phenylalanine |
| PiOH | H₃O₄P | Phosphate |
| PPiOH | H₄O₇P₂ | Pyrophosphate |
| Pro | C₅H₉O₂N | Proline |
| Prot | C_{4.8}H_{7.67}O_{1.4}N_{1.37} | Protein composition |
| | S_{0.046} | |
| ProtoCat | C₇H₆O₄ | Protocatechol |
| PRPP | C₅H₁₃O₁₄P₃ | 5-phospho-alpha-D-ribosyl-1-pyrophosphate |
| PSer | C₄₀H₇₆O₁₀NP | Phosphatidyl Serine |
| Pyr | C₃H₄O₃ | Pyruvate |
| Qa | C₇H₁₂O₆ | Quinate |
| R1P | C₅H₁₁O₈P | Ribose-1-phosphate |
| R5P | C₅H₁₁O₈P | Ribose-5-phosphate |
| Rl5P | C₅H₁₁O₈P | Ribulose-5-phosphate |
| RNA | C_{9.58}H_{13.8}O_{7.95}N_{3.95}P | RNA composition |
| S7P | C₇H₁₅O₁₀P | Sedoheptulose-7-phosphate |
| Ser | C₃H₇O₃N | Serine |
| Shi | C₇H₁₀O₅ | Shikimate |
| Shi3P | C₇H₁₁O₈P | Shikimate-3-phosphate |
| Suc | C₄H₆O₄ | Succinate |
| SucCoA | C₂₅H₃₆O₁₉N₇P₃S | Succinyl CoA |
| THF | C₁₉H₂₃O₆N₇ | Tetrahydrofolate |
| Thiored | Pept | Thioredoxin |
| ThioredH2 | H₂Pept | Reduced thioredoxin |
| Thr | C₄H₉O₃N | Threonine |
| Trp | C₁₁H₁₂O₂N₂ | Tryptophan |
| Tyr | C₉H₁₁O₃N | Tyrosine |
| UDP | C₉H₁₄O₁₂N₂P₂ | Uridine diphosphate |
| UDPGlc | C₁₅H₂₄O₁₇N₂P₂ | UDP glucose |
| UDPNAG | C₁₇H₂₇O₁₇N₃P₂ | UDP N-acetyl glucosamine |
| UMP | C₉H₁₃O₉N₂P | Uridine monophosphate |
| UTP | C₉H₁₅O₁₅N₂P₃ | Uridine triphosphate |
| Val | C₅H₁₁O₂N | Valine |
| XMP | C₁₀H₁₃O₉N₄P | Xanthosine-5-phosphate |
| Xu5P | C₅H₁₁O₈P | Xylulose-5-phosphate |

### REFERENCES

1. Postma, P. W., Lengeler, J. W., and Jacobson, G. R. (1993) Microbiological Reviews 57(3), 543-594
2. Paulsen, I. T., Nguyen, L., Sliwinski, M. K., Rabus, R., and Saier, M. H. (2000) J. Mol. Biol. 301(1), 75-100
3. Paulsen, I. T., Sliwinski, M. K., and Saier, M. H. (1998) J. Mol. Biol. 277(3), 573-592
4. Werpy, T., Petersen, G., Aden, A., Bozell, J., Holladay, J., White, J., and Manheim, A. (2004) Top Value Added Chemicals from Biomass. Volume I: Results of Screening for Potential Candidates from Sugar and Synthesis Gas. In., US Department of Energy, Oak Ridge, USA
5. McKinlay, J. B., Vieille, C., and Zeikus, J. G. (2007) Applied Microbiology and Biotechnology 76(4), 727-740
6. Patel, M., Crank, M., Dornburg, V., Hermann, B., Roes, L., Husing, B., Overbeek, L., Terragni, F., and Recchia, E. (2006) Medium and Long-term Opportunities and Risks of the Biotechnological Production of Bulk Chemicals from Renewable Resources—The Potential of White biotechnology. In. The Brew report, University of Utrecht, Utrecht
7. Zeikus, G. J., Jain, M. K., and Elankovan, P. (1999) Applied Microbiology and Biotechnology 51(5), 545-552
8. Janausch, I. G., Zientz, E., Tran, Q. H., Kroger, A., and Unden, G. (2002) Biochimica Et Biophysica Acta-Bioenergetics 1553(1-2), 39-56
9. Saier, M. H., Tran, C. V., and Barabote, R. D. (2006) Nucleic Acids Research 34, D181-D186
10. Clark, D. P. (1989) Fems Microbiology Reviews 63(3), 223-234
11. Davies, S. J., Golby, P., Omrani, D., Broad, S. A., Harrington, V. L., Guest, J. R., Kelly, D. J., and Andrews, S. C. (1999) Journal of Bacteriology 181(18), 5624-5635
12. Soupene, E., van Heeswijk, W. C., Plumbridge, J., Stewart, V., Bertenthal, D., Lee, H., Prasad, G., Paliy, O., Charernnoppakul, P., and Kustu, S. (2003) Journal of Bacteriology 185(18), 5611-5626
13. Datsenko, K. A., and Wanner, B. L. (2000) Proceedings Of The National Academy Of Sciences Of The United States Of America 97(12), 6640-6645
14. Lequeux, G., Johansson, L., Maertens, J., Vanrolleghem, P., and Liden, G. (2005) Journal of Biotechnology 118, S121-S121
15. Majewski, R. A., and Domach, M. M. (1990) Biotechnology and Bioengineering 35(7), 732-738
16. Varma, A., and Palsson, B. O. (1993) Journal of Theoretical Biology 165(4), 503-522
17. R-Development Core Team (2006).R: A Language and Environment for Statistical Computing. R Foundation for Statistical Computing. Vienna, Austria
18. Wold, S., Sjostrom, M., and Eriksson, L. (2001) Chemometrics and Intelligent Laboratory Systems 58(2), 109-130
19. von Kamp, A., and Schuster, S. (2006) Bioinformatics 22(15), 1930-1931

1. A mutant and/or recombinant micro-organism comprising a genetic change leading to increased succinate export activity and decreased succinate import activity.

2. The mutant and/or recombinant micro-organism according to claim 1 wherein said micro-organism is an Escherichia coli strain.

3. The mutant and/or recombinant micro-organism according to claim 1, wherein said genetic change affects the dcuC exporter gene and the dctA importer gene.

4. The mutant and/or recombinant micro-organism according to claim 3, wherein said genetic change is the replacement of the promoter of the dcuC exporter gene and the knock-out of dctA importer gene.

5. The mutant and/or recombinant micro-organism of claim 1, further comprising:
a genetic change in one or more of the genes selected from the group consisting of ackA, poxB, pta, arcA, sdhA, sdhB, sdhC, sdhD, iclR, citD, citE, citF, pckA, maeA, maeB, eda, edd, gltA, ppc, sstT, ydjN, ygjE, citT/ybdS, ybhI, yJbS, yhjE and ydf.

6. A process for producing succinate, wherein the improvement comprises:
utilizing a mutant and/or recombinant micro-organism comprising a genetic change leading to increased succinate export activity and/or decreased succinate import activity, in combination with a genetic change leading to increased succinate production to produce succinate.

7. A process for producing a succinate, wherein the improvement comprises:
utilizing the mutant and/or recombinant micro-organism of claim 1 to produce succinate.

8. The process according to claim 6, wherein said process is under aerobic conditions.

9. The mutant and/or recombinant microorganism of claim 2, wherein the genetic change alters the dcuC gene and the dctA gene.

10. The mutant and/or recombinant microorganism of claim 9, wherein the genetic change comprises replacing the promoter of the dcuC gene and knocking-out the dctA gene.

11. The mutant and/or recombinant microorganism of claim 10, further comprising:
a genetic change in one or more of the genes selected from the group consisting of ackA, poxB, pta, arcA, sdhA, sdhB, sdhC, sdhD, iclR, citD, citE, citF, pckA, maeA, maeB, eda, edd, gltA, ppc, sstT, ydjN, ygjE, citT/ybdS, ybhI, yfbS, yhjE, and ydfJ.

12. The mutant and/or recombinant microorganism of claim 4, further comprising:
a genetic change in one or more of the genes selected from the group consisting of ackA, poxB, pta, arcA, sdhA, sdhB, sdhC, sdhD, iclR, citD, citE, citF, pckA, maeA, maeB, eda, edd, gltA, ppc, sstT, ydjN, ygjE, citT/ybdS, ybhI, yfbS, yhjE, and ydfJ.

13. The process of claim 7, wherein the process is conducted under aerobic conditions.

14. A process for producing a succinate, wherein the improvement comprises:
utilizing the mutant and/or recombinant microorganism of claim 2 to produce succinate.

15. The process of claim 14, wherein the process is conducted under aerobic conditions.

16. A process for producing a succinate, wherein the improvement comprises:
utilizing the mutant and/or recombinant microorganism of claim 3 to produce succinate.

17. The process of claim 16, wherein the process is conducted under aerobic conditions.

18. A process for producing succinate, wherein the improvement comprises:
utilizing the mutant and/or recombinant microorganism of claim 2 to produce succinate.

19. The process of claim 18, wherein the process is conducted under aerobic conditions.

20. A bacterial strain of the type having a dcuC gene and a dctA gene, wherein the bacterial strain is isolated, mutant, and/or recombinant, the improvement comprising:
replacing the promoter of the dcuC gene and knocking-out the dctA gene of the bacterial strain so as to increase succinate export activity and decrease succinate import activity of the bacterial strain.
